(19) United States
(12) Patent Application Publication
(10) Pub. No.: US 2018/0263588 A1
(43) Pub. Date: Sep. 20, 2018

(54) DYNAMIC RADIOGRAPHIC IMAGE PROCESSING APPARATUS

(71) Applicant: Konica Minolta, Inc., Tokyo (JP)

(72) Inventor: Koichi FUJIWARA, Tokyo (JP)

(21) Appl. No.: 15/869,832

(22) Filed: Jan. 12, 2018

(30) Foreign Application Priority Data
Mar. 16, 2017 (JP) 2017-051036

(51) Int. Cl. A61B 6/00; G06T 7/246; G06T 5/00; G06T 7/00

(52) U.S. Cl. CPC A61B 6/5264; G06T 7/248; G06T 2207/30004; G06T 7/0016; G06T 2207/10116; G06T 5/003

(57) ABSTRACT

A dynamic radiographic image processing apparatus includes: an estimator that extracts a first image of an anatomical region from a first frame image and a second image of the same anatomical region as the first image from a second frame image, and estimates a quantity of motion of a body from a change from the first image to the second image, the first frame image and the second frame image being included in a dynamic radiographic image obtained by radiographic imaging of the body in a respiratory state, motion accompanying respiration not appearing in the first image and the second image; and an image converter that performs image conversion on the second frame image to suppress influence of motion of the body, using the quantity of motion.

The entire disclosure of Japanese patent Application No. 2017-051036, filed on Mar. 16, 2017, is incorporated herein by reference in its entirety.

### BACKGROUND

### Technological Field

The present invention relates to a dynamic radiographic image processing apparatus.

### Description of the Related Art

A dynamic radiographic image that shows motion of or a change in the target site can be easily obtained these days by radiographic imaging utilizing digital technology. For example, a dynamic radiographic image that shows the target site to be tested or examined can be easily obtained by radiographic imaging using a semiconductor image sensor such as a flat X-ray detector (flat panel detector (FPD)).

However, in a case where motion is caused in the body while radiographic imaging is being performed, a temporal change reflecting the motion appears in the dynamic radiographic image, and this hinders analysis of the dynamic radiographic image. For example, analysis to be conducted by extracting only ventilation components is hindered.

A technology disclosed in JP 3919799 B2 aims to solve this problem. By the technology disclosed in JP 3919799 B2, each image is shifted to the thorax position serving as the reference position (paragraph [0034] in JP 3919799 B2).

By the technology disclosed in JP 3919799 B2, each image is shifted to the thorax position serving as the reference position. However, motion occurs with respiration in the thorax, and the motion accompanying respiration also appears in an image of the thorax.

Therefore, by the technology disclosed in JP 3919799 B2, positioning is not appropriately performed, and motion caused in the body during radiographic imaging is not effectively suppressed from hindering analysis of a dynamic radiographic image.

### SUMMARY

The present invention has been made to solve the above problem. An object of the present invention is to suppress motion caused in the body during radiographic imaging from hindering analysis of a dynamic radiographic image.

To achieve the abovementioned object, according to an aspect of the present invention, a dynamic radiographic image processing apparatus reflecting one aspect of the present invention comprises: an estimator that extracts a first image of an anatomical region from a first frame image and a second image of the same anatomical region as the first image from a second frame image, and estimates a quantity of motion of a body from a change from the first image to the second image, the first frame image and the second frame image being included in a dynamic radiographic image obtained by radiographic imaging of the body in a respiratory state, motion accompanying respiration not appearing in the first image and the second image; and an image converter that performs image conversion on the second frame image to suppress influence of motion of the body, using the quantity of motion.

### BRIEF DESCRIPTION OF THE DRAWINGS

The objects, advantages, aspects, and features provided by one or more embodiments of the invention will become more fully understood from the detailed description given hereinbelow and the appended drawings which are given by way of illustration only, and thus are not intended as a definition of the limits of the present invention:

FIG. 1 is a block diagram showing a dynamic radiographic image capturing/processing system according to a first embodiment;

FIG. 2 is a diagram schematically showing a dynamic radiographic image generated in the dynamic radiographic image capturing/processing system according to the first embodiment;

FIG. 3 is a flowchart showing the flow of processing in the dynamic radiographic image capturing/processing system according to the first embodiment;

FIG. 4 is a diagram schematically showing the body being imaged by the dynamic radiographic image capturing/processing system according to the first embodiment;

FIG. 5 is a diagram schematically showing a dynamic radiographic image generated in the dynamic radiographic image capturing/processing system according to the first embodiment;

FIG. 6 is a diagram schematically showing an example of a change in a spine image in a case where the body has moved forward in the dynamic radiographic image capturing/processing system according to the first embodiment;

FIG. 7 is a diagram schematically showing an example of a change in a spine image in a case where the body has moved backward in the dynamic radiographic image capturing/processing system according to the first embodiment;

FIG. 8 is a diagram schematically showing an example of a change in a spine image in a case where the body has rotated about an axis extending in the imaging direction in the dynamic radiographic image capturing/processing system according to the first embodiment;

FIG. 9 is a diagram schematically showing an example of a change in a spine image in a case where the body has rotated about an axis extending in the body width direction in the dynamic radiographic image capturing/processing system according to the first embodiment; and

FIG. 10 is a diagram schematically showing an example of changes in a spine image and a rib image in a case where the body has rotated about an axis extending in the body height direction in the dynamic radiographic image capturing/processing system according to the first embodiment.

### DETAILED DESCRIPTION OF EMBODIMENTS

Hereinafter, one or more embodiments of the present invention will be described with reference to the drawings. However, the scope of the invention is not limited to the disclosed embodiments.

1. Dynamic radiographic image capturing/processing system

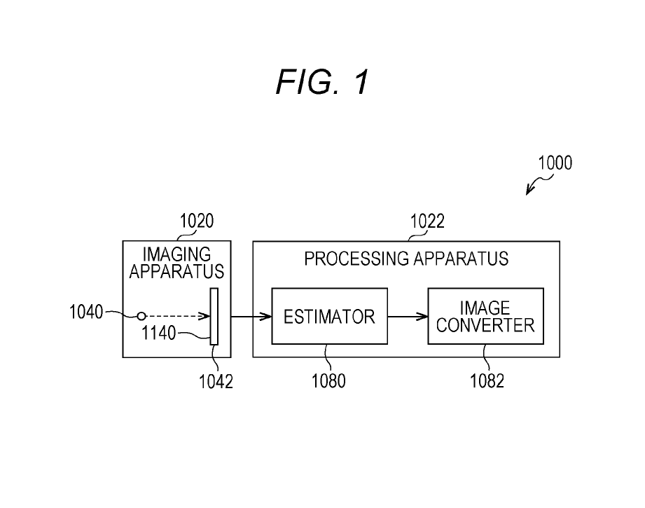

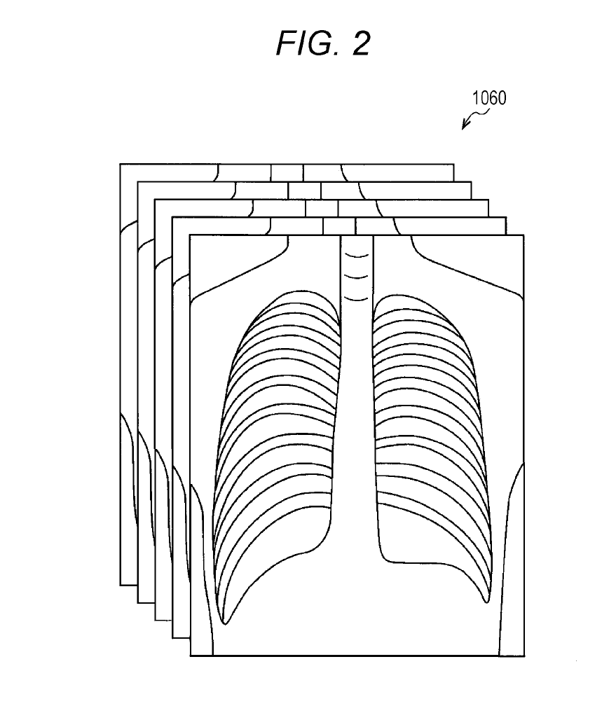

FIG. 1 is a block diagram showing a dynamic radiographic image capturing/processing system according to a first embodiment. FIG. 2 is a diagram showing a dynamic radiographic image generated in the dynamic radiographic image capturing/processing system according to the first embodiment.

The dynamic radiographic image capturing/processing system 1000 shown in FIG. 1 includes an imaging apparatus 1020 and a processing apparatus 1022.

The imaging apparatus 1020 includes an X-ray source 1040 and a flat X-ray detector (a flat panel detector (FPD)) 1042, and generates a dynamic radiographic image 1060 shown in FIG. 2.

In one radiographic imaging operation, the imaging apparatus 1020 generates X-rays from the X-ray source 1040, causes the generated X-rays to penetrate through the human body, and detects the X-rays having penetrated through the human body with the FPD 1042. By doing so, the imaging apparatus 1020 generates a frame image including images of various anatomical regions in the body in the one radiographic imaging operation. By performing radiographic imaging twice or more, the imaging apparatus 1020 generates the dynamic radiographic image 1060 including two or more frame images. The dynamic radiographic image 1060 is also called a radiographic dynamic image. The dynamic radiographic image capturing/processing system 1000 is designed to image the chest, and generate the chest dynamic radiographic image 1060. The chest dynamic radiographic image 1060 is to be used in dynamic analysis of the pulmonary filed, such as ventilation analysis or analysis of the bloodstream in the pulmonary field.

The processing apparatus 1022 includes an estimator 1080 and an image converter 1082, and processes the generated dynamic radiographic image 1060.

The estimator 1080 extracts a spine image from a reference frame image included in the generated dynamic radiographic image 1060, extracts a spine image from each frame image among the frame images included in the dynamic radiographic image 1060, and estimates quantities of motion of the body from the changes from the spine image extracted from the reference frame image to the spine images extracted from the respective frame images.

The spine is an anatomical region in which no motion occurs with respiration. Therefore, no motion accompanying respiration appears in a spine image. However, motion accompanying body motion appears in the spine image. Accordingly, a quantity of body motion can be estimated from a change in the spine image, without being affected by motion accompanying respiration.

Instead of spine images, images of an anatomical region other than the spine may be extracted. The anatomical region images to be extracted are selected so that motion accompanying respiration does not appear in the images but motion accompanying body motion appears in the images. Even in a case where motion accompanying respiration appears in an anatomical region as an object, if motion accompanying respiration does not appear in images of the anatomical region, the images of the anatomical region may be extracted.

Using the estimated quantities of motion, the image converter 1082 performs image conversion on each frame image. The image conversion is performed so that the influence of body motion is suppressed.

2. Processing flow

2.1. Acquisition of a dynamic radiographic image

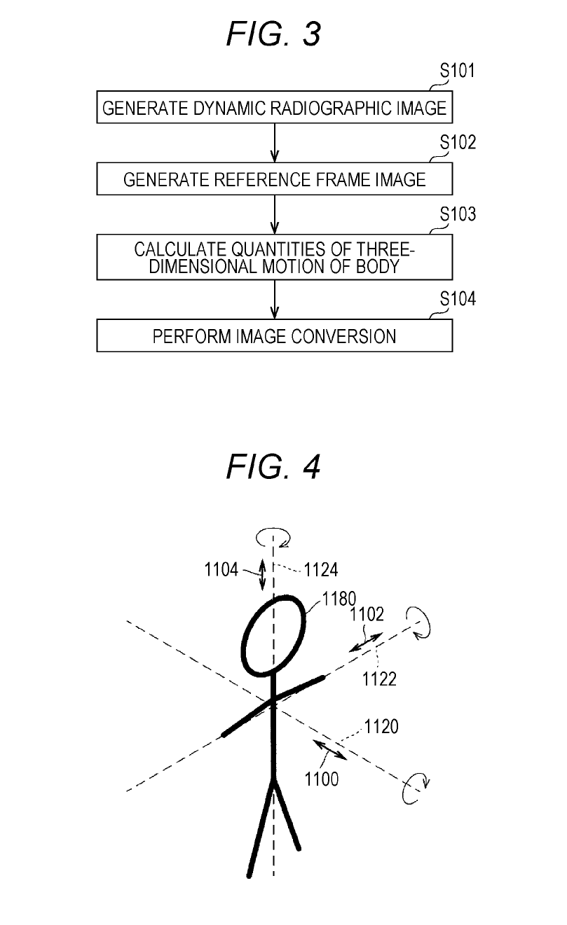

FIG. 3 is a flowchart showing the flow of processing in the dynamic radiographic image capturing/processing system according to the first embodiment.

In step S101 shown in FIG. 3, the imaging apparatus 1020 generates the dynamic radiographic image 1060. The generated dynamic radiographic image 1060 is input to the estimator 1080.

2.2. Determination of the reference frame image

In step S102, the estimator 1080 determines the reference frame image. The reference frame image is one of the frame images included in the input dynamic radiographic image 1060, and is the frame image captured first or last among the frame images, for example. Alternatively, the respiratory state is analyzed, and a frame image of a certain respiratory phase may be used as the reference frame image. For example, a frame image of a resting expiratory level or a resting inspiratory level may be used as the reference frame image.

2.3. Calculation of three-dimensional motion quantities

In step S103, the estimator 1080 calculates a quantity of three-dimensional motion of the body from the position and the posture of the body at the time when the reference frame image was captured to the position and the posture of the body at the time when each frame image was captured. A quantity of three-dimensional motion is a quantity indicating the amount of body motion that does not fall in one plane.

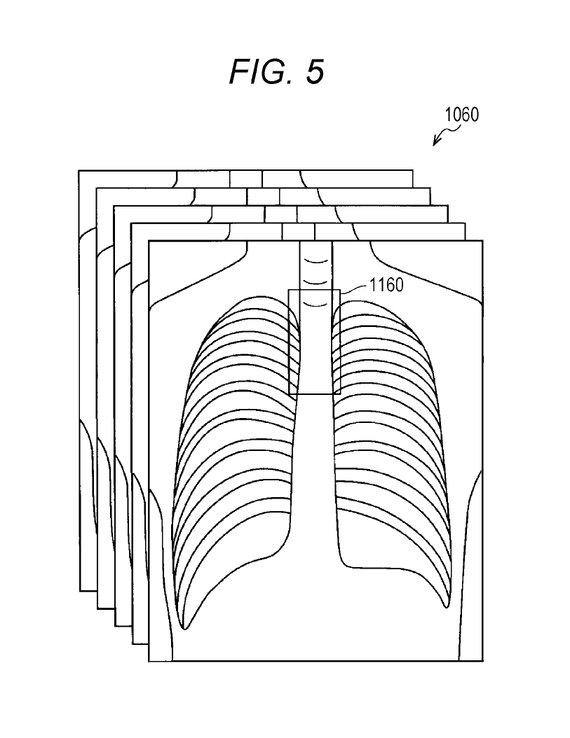

FIG. 4 is a diagram schematically showing the body being imaged by the dynamic radiographic image capturing/processing system according to the first embodiment. FIG. 5 is a diagram showing a dynamic radiographic image generated in the dynamic radiographic image capturing/processing system according to the first embodiment.

A three-dimensional motion quantity to be calculated includes translation in the imaging direction indicated by an arrow 1100 shown in FIG. 4, rotation about an axis 1120 extending in the imaging direction, rotation about an axis 1122 extending in the body width direction that is perpendicular to the imaging direction and is indicated by an arrow 1102 shown in FIG. 4, and rotation about an axis 1124 extending in the body height direction that is perpendicular to the imaging direction and is indicated by an arrow 1104 shown in FIG. 4. The imaging direction is perpendicular to the imaging area 1140 of the FPD 1042. Quantities of motion other than the above quantities of motion may also be calculated. Alternatively, only the quantity of motion that has the largest influence on analysis among these quantities of motion may be calculated. Instead of quantities of three-dimensional motion, quantities of one-dimensional or two-dimensional motion may be calculated.

As shown in FIG. 5, in the calculation of quantities of three-dimensional motion, reference regions 1160 in which a spine image exists are extracted from the reference frame image and the respective frame images, and quantities of three-dimensional motion of the body are calculated from the changes from the reference region 1160 extracted from the reference frame image to the reference regions 1160 extracted from the respective frame images. In some cases, instead of the reference regions 1160 in which a spine image exists, reference regions in which an image of the spine and the ribs exists are extracted.

2.4. Calculation of translation in the imaging direction

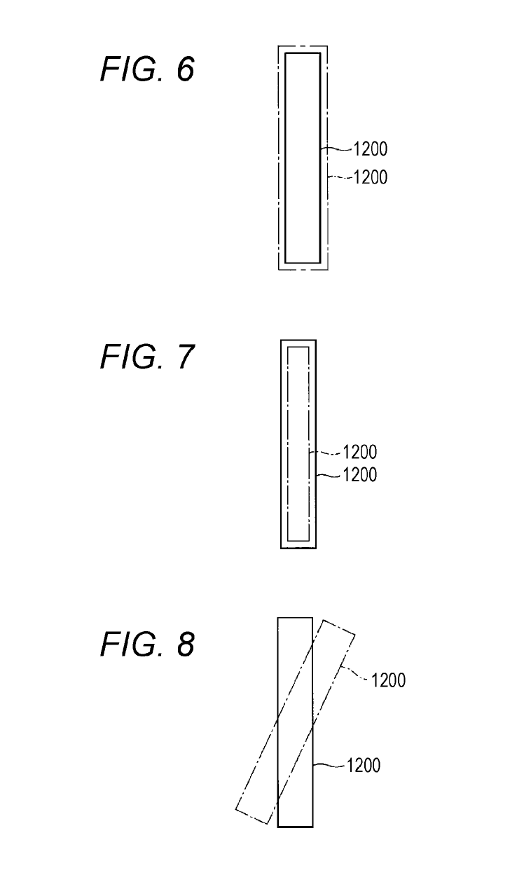

FIG. 6 is a diagram schematically showing an example of a change in a spine image in a case where the body has moved forward in the dynamic radiographic image capturing/processing system according to the first embodiment. FIG. 7 is a diagram schematically showing an example of a change in a spine image in a case where the body has moved backward in the dynamic radiographic image capturing/processing system according to the first embodiment.

In the case where a body 1180 has moved forward to become closer to the FPD 1042, a spine image 1200 is enlarged in the dynamic radiographic image 1060 as shown in FIG. 6. In the case where the object has moved backward to become further away from the FPD 1042, the spine image 1200 is reduced in the dynamic radiographic image 1060 as shown in FIG. 7.

Therefore, the translation in the imaging direction is calculated from the enlargement or the reduction from the reference region 1160 extracted from the reference frame image to the reference region 1160 extracted from each frame image. For example, translation Lx(R-1) in the imaging direction is calculated from an enlargement factor R that is the ratio of the size of the reference region 1160 extracted from each frame image to the size of the reference region 1160 extracted from the reference frame image, and the distance L from the X-ray source 1040 to the FPD 1042.

2.5. Calculation of rotation about the axis extending in the imaging direction

FIG. 8 is a diagram schematically showing an example of a change in a spine image in a case where the body has rotated about the axis extending in the imaging direction in the dynamic radiographic image capturing/processing system according to the first embodiment.

In a case where the body 1180 has rotated about the axis 1120 extending in the imaging direction, the spine image 1200 rotates in the dynamic radiographic image 1060 as shown in FIG. 8. This rotation of the spine image 1200 is conspicuous. Therefore, the rotation about the axis 1120 extending in the imaging direction is calculated from the rotation from the reference region 1160 extracted from the reference frame image to the reference region 1160 extracted from each frame image. For example, the reference region 1160 extracted from the reference frame image is rotated small angles θ1, θ2, . . . , and θn, and reference regions RG1, RG2, . . . , and RGn are obtained after the rotation. The reference regions RG1, RG2, . . . , and RGn obtained after the rotation are matched against the reference regions 1160 extracted from the respective frame images, and the small angle θi, which gives the highest correlation, is selected from among the small angles θ1, θ2, . . . , and θn. The selected small angle θi is regarded as the rotation about the axis 1120 extending in the imaging direction. This matching is matching in a two-dimensional space, but rotation in a two-dimensional space matches rotation in a three-dimensional space.

2.6. Calculation of rotation about the axis extending in the body width direction

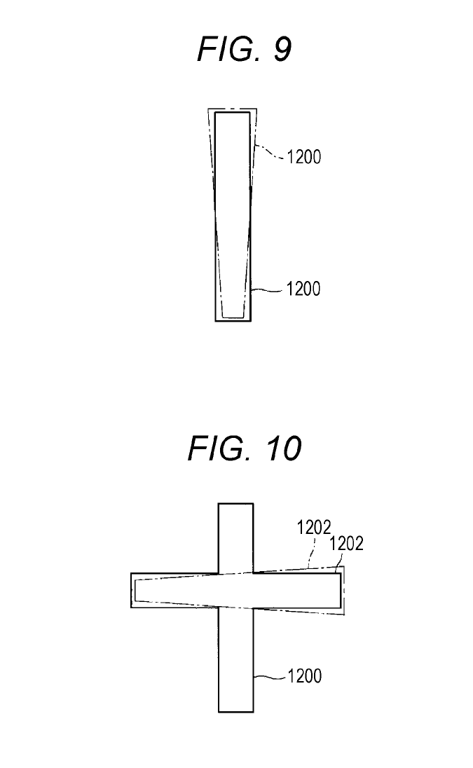

FIG. 9 is a diagram schematically showing an example of a change in a spine image in a case where the body has rotated about the axis extending in the body width direction in the dynamic radiographic image capturing/processing system according to the first embodiment.

In a case where the body 1180 has rotated about the axis 1122 extending in the body width direction, the spine image 1200 is deformed in the dynamic radiographic image 1060 as shown in FIG. 9. This deformation of the spine image 1200 is inconspicuous. Therefore, a three-dimensional (3D) model of the spine is prepared, and rotation about the axis 1122 extending in the body width direction is calculated with the use of the prepared 3D model. For example, the prepared 3D model is rotated small angles θ1, θ2, . . . , and θn, and, as a result, 3D models MD1, MD2, . . . , and MDn are obtained after the rotation. The 3D models MD1, MD2, . . . , and MDn obtained after the rotation are then projected onto a flat surface, and, as a result, projection images P11, P12, . . . , and Pln are obtained. The projection images P11, P12, . . . , and Pln are matched against the reference regions 1160 extracted from the respective frame images, and the small angle θi, which gives the highest correlation, is selected from among the small angles θ1, θ2, . . . , and θn. The selected small angle θi is regarded as the rotation about the axis 1122 extending in the body width direction.

2.7. Calculation of rotation about the axis extending in the body height direction

FIG. 10 is a diagram schematically showing an example of changes in a spine image and a rib image in a case where the body has rotated about the axis extending in the body height direction in the dynamic radiographic image capturing/processing system according to the first embodiment.

In a case where the body 1180 has rotated about the axis 1124 extending in the body height direction, the spine image 1200 is hardly deformed but a rib image 1202 is deformed in the dynamic radiographic image 1060. Therefore, a reference region in which an image of the spine and an image of the ribs existing near the spine exist is extracted, and the rotation about the axis 1124 extending in the body height direction is calculated in the same manner as in the case where the rotation about the axis 1122 extending in the body width direction is calculated. In a case where the body 1180 has rotated about the axis 1122 extending in the body width direction, the spine image does not change conspicuously, but the rib image changes to a certain extent. Accordingly, in a case where the reference region is a region in which an image of the spine and an image of the ribs exist, the rotation about the axis 1124 extending in the body height direction is appropriately calculated.

2.8. Image conversion

In step S104, the image converter 1082 performs image conversion on the respective frame images, using the calculated quantities of three-dimensional motion. In the image conversion, positioning is performed to adjust each frame image to the reference frame image. For example, a determinant for converting images is obtained from the calculated quantities of three-dimensional motion, and calculation using the obtained determinant is performed on each frame image.

With the dynamic radiographic image capturing/processing system 1000 according to the first embodiment, motion caused in the body 1180 during radiographic imaging can be suppressed from hindering analysis of the dynamic radiographic image 1060.

Although embodiments of the present invention have been described and illustrated in detail, the disclosed embodiments are made for purposes of illustration and example only and not limitation. The scope of the present invention should be interpreted by terms of the appended claims. It should be understood that numerous modifications not mentioned herein can be made without departing from the scope of the invention.

What is claimed is:

1. A dynamic radiographic image processing apparatus comprising:
an estimator that extracts a first image of an anatomical region from a first frame image and a second image of the same anatomical region as the first image from a second frame image, and estimates a quantity of motion of a body from a change from the first image to the second image, the first frame image and the second frame image being included in a dynamic radiographic image obtained by radiographic imaging of the body in a respiratory state, motion accompanying respiration not appearing in the first image and the second image; and
an image converter that performs image conversion on the second frame image to suppress influence of motion of the body, using the quantity of motion.

2. The dynamic radiographic image processing apparatus according to claim 1,
wherein the quantity of motion is a quantity of three-dimensional motion.

3. The dynamic radiographic image processing apparatus according to claim 1,
wherein the change includes at least one of enlargement, reduction, rotation, and deformation.

4. The dynamic radiographic image processing apparatus according to claim 1,
wherein the anatomical region includes at least one of a spinal region and a costal region.
